(19) United States
(12) Patent Application Publication
(10) Pub. No.: US 2015/0239832 A1
(43) Pub. Date: Aug. 27, 2015

(54) ANALOGS AND PRODRUGS OF LOOP DIURETICS, INCLUDING BUMETANIDE, FUROSEMIDE AND PIRETANIDE; COMPOSITIONS AND METHODS OF USE

(71) Applicant: Neuro Pro Therapeutics, Inc., Bahama, NC (US)

(72) Inventors: Daryl W. Hochman, Bahama, NC (US); Matthew David Orr, Raleigh, NC (US); Mustapha Soukri, Raleigh, NC (US)

(21) Appl. No.: 14/423,099

(22) PCT Filed: Sep. 3, 2013
(86) PCT No.: PCT/US13/57885

§ 371 Date: Feb. 20, 2015

Related U.S. Application Data
(60) Provisional application No. 61/696,760, filed on Sep. 4, 2012.

(51) Int. Cl. C07C 311/39; C07D 207/09; C07D 311/00; C07D 495/08; C07D 221/22; C07D 471/08; C07D 491/08; C07D 307/52; C07D 335/04

(52) U.S. Cl. CPC C07C 311/39; C07D 307/52; C07D 207/09; C07D 311/00; C07D 335/04; C07D 221/22; C07D 471/08; C07D 491/08; C07D 495/08

(57) ABSTRACT

Novel analogs and prodrugs of the loop diuretics bumetanide, furosemide and piretanide are described. Pharmaceutical compositions containing loop diuretic analogs and prodrugs are also described. These analogs and prodrugs are particularly useful for the treatment and/or prophylaxis of conditions that involve the NKCC cotransporter family (NKCC1 and NKCC2), or the KCC cotransporter family (KCC1, KCC2, KCC3, KCC4), or GABAa receptors. Such conditions include, but are not limited to anxiety disorders, epilepsy, migraine, non-epileptic seizures, sleep disorders, obesity, eating disorders, autism, depression, edema, glaucoma, stroke, ischemia, neuropathic pain, addictive disorders, schizophrenia, psychosis, and tinnitus.

### REFERENCE TO RELATED APPLICATION

This application claims priority to provisional U.S. patent application Ser. No. 61/696,760 filed Sep. 4, 2012. The disclosure of this priority patent application is incorporated herein by reference in its entirety.

### TECHNICAL FIELD OF INVENTION

The present disclosure relates to chemical analogs and prodrugs of the loop diuretics bumetanide, furosemide and piretanide. Furthermore, the present disclosure relates to the use of methods and compositions of analogs and prodrugs of bumetanide, furosemide and piretanide for treatment of neurological and psychiatric disorders by administering these agents that modulate expression and/or activity of ion transporters of the NKCC family, and/or the KCC family, and/or GABAa-mediated synaptic signaling.

### BACKGROUND OF THE INVENTION

General

Many of the agents that are currently used to treat neurological and psychiatric disorders are thought to mediate their therapeutic effects by modulating the excitability of neurons, or some aspect of synaptic signaling between neurons, in the nervous system. Such therapeutic agents, however, affect every cell in the brain indiscriminately, regardless of whether or not the cell contributes to the neurological or psychiatric disorder. In other words, the normal functions of normal cells are affected by these treatments, as are the abnormal functions of cells that underlie the pathological condition being treated. As a consequence, treatments used to treat most neurological and psychiatric disorders elicit unwanted neurological and cognitive side effects. The methods and compositions of the present invention avoid these side effects, since they mediate their therapeutic effects by modulating ion cotransporters on neurons and glia, and do not have effects on ion channels or excitatory synaptic transmission (Hochman, Epilepsia, 2012).

Anxiety

Anxiety disorders are the most prevalent class of psychiatric conditions, affecting approximately 18% of adults [1]-[3]. These disorders include Panic Disorder (PD), Social Anxiety Disorder (SAD), Obsessive Compulsive Disorder (OCD), Posttraumatic Stress Disorder (PTSD), Generalized Anxiety Disorder (GAD), and Specific Phobia [4]. Medications currently used for treating these disorders include tricyclic antidepressants, selective serotonin reuptake inhibitors (SSRIs), serotonin norepinephrine reuptake inhibitors (SNRIs), benzodiazepines, anticonvulsants, and monoamine oxidase inhibitors. However, 20%-40% of anxiety patients remain non-responders to all available therapies [5]. Additionally, many of the anxiolytic medications can elicit central nervous system (CNS) side-effects that patients find difficult to tolerate [5], [6]. There is a need for new pharmacotherapeutic approaches to treat anxiety with greater efficacy and fewer side effects.

γ-aminobutyric acid (GABA) is the primary inhibitory neurotransmitter in the CNS. The downregulation of GABA_{A} inhibition in the brain has been hypothesized to contribute to pathophysiological anxiety [7]. Antiepileptic drugs that enhance GABA_{A} signaling often possess anxiolytic properties and are commonly prescribed to treat anxiety. These drugs include pregabalin for GAD, pregabalin and gabapentin for SAD, and a number of benzodiazepines for GAD, SAD, and panic disorder [8]. The loop diuretics furosemide (Lasix) and bumetanide (Bumex) are also thought to be GABA_{A} modulators with antiepileptic properties [9]-[12]. These drugs have attracted some interest from epilepsy researchers because of their antiepileptic effects over a wide variety of experimental seizure models [9], [11], [13], [14], and several clinical findings suggesting they can suppress seizures in patients with medically intractable epilepsy [15], [16].

Loop diuretics are thought to affect GABA_{A} dependent signaling in the brain through their antagonism of cation-chloride cotransport, which is a distinctly different mechanism of action from all other known pharmacological GABA_{A} modulators [17]. Specifically, furosemide and bumetanide antagonize the Na⁺-K⁺-2Cl⁻ (NKCC1) cotransporter that is present on both neurons and glial cells, and the neuron-specific K⁺-Cl⁻ (KCC2) cotransporter [10], [11], [18]-[20]. NKCC1 normally transports chloride from the extracellular to intracellular spaces, and KCC2 transports chloride from intracellular to extracellular spaces. Although furosemide and bumetanide are thought to antagonize both cotransporters, they both have significantly greater affinity for NKCC1 over KCC2 [10]. Hyperpolarizing inhibitory postsynaptic potentials in neurons are generated by the influx of anions (HCO₃⁻ and Cl⁻) down their electrochemical gradients [21]. Since GABA_{A} receptor-mediated current is determined, in part, by the difference between the equilibrium potential for Cl⁻ and the neuronal membrane potential [22], preferential antagonism of NKCC1 with a loop diuretic would be expected to cause a hyperpolarizing shift in the GABA reversal potential, enhancing GABA_{A} synaptic signalling. This effect can be particularly important in view of recent work showing the dominant role that NKCC1 plays at the axon initial segment of principal neurons [23], [24].

It has recently been shown the furosemide and bumetanide significantly reduce conditioned anxiety in the contextual fear-conditioning and fear-potentiated startle rat models of anxiety. Krystal et al., Loop diuretics Have Anxiolytic Effects in Rat Models of Conditioned Anxiety, PLoS ONE Vol. 7 Issue 4 e35417, April 2012.

Epilepsy

It has long been hypothesized that volume and ion changes in the extracellular space (ECS) can modulate the excitability and epileptogenicity of tissue (Andrew, 1991; Jefferys, 1995; Dudek et al., 1998). Neuronal networks interact with the surrounding ECS in a dynamic, feedback-loop manner. Action potential firing can change the ion concentrations and volume of the ECS, and likewise these changes in the ECS are thought to modulate synaptic transmission and neuronal excitability (Hochman, 2009). The proportion of a volume of brain tissue that is composed of the ECS is called the extracellular volume fraction (EVF). The EVF is a dynamic entity that can change within localized microscopic regions in response to neuronal activity. Action potential firing and synaptic activity generate localized increases in extracellular potassium and chloride. These ion gradients are dispersed, in part, via movement into glial cells through membrane-bound ion transporters and channels (Sontheimer, 1994; Chen & Nicholson, 2000; Emmi et al., 2000; Simard & Nedergaard, 2004). These changing ion concentrations generate osmotic gradients between extracellular and intracellular compartments, causing the diffusion of water into hypertonic spaces. The end result is an activity-driven movement of water from intracellular compartments into glial cells, mediating a transient reduction of the EVF through glial cell swelling (Simard & Nedergaard, 2004; Østby et al., 2009). These considerations suggest that the microscopic organization of glial cell processes could potentially contribute significantly to the ionic and volume changes of the ECS. An electron microscopy study showed that glial cell processes proliferate within specific microdomains in response to increases in neuronal activity during the induction of long-term potentiation (LTP) (Wenzel et al., 1991). It may be that epileptiform activity also alters the distribution of astrocytic processes in ways that are important in epileptogenesis.

The loop diuretics are known to modulate ion cotransporters on neurons and glia in the brain, including a neuronal isoform of the KCC2 and the Na+-K-2Cl cotransporter (NKCC1) that is present on both neurons and glia (Russel, 2000; Blaesse et al., 2009). Under normal physiologic conditions, KCC2 transports K+ and Cl− from the intracellular spaces of neurons into the ECS, and NKCC1 transports Na+, K+, and Cl− from the ECS into the intracellular spaces of neurons and glia. The loop diuretics, furosemide (Lasix) and bumetanide (Bumex) are classic NKCC1 antagonists, with bumetanide being a more potent and specific antagonist than furosemide (Russel, 2000). Reduction of extracellular chloride (low-[Cl⁻]ₒ) by equimolar substitution with impermeant anions such as gluconate, also antagonizes NKCC1. Furosemide antagonizes KCC2 in addition to NKCC1, and can thus reduce γ-aminobutyric acid receptor A (GABA_{A}) inhibition in adult neurons by reducing the neuronal transmembrane chloride gradient (Thompson et al., 1988). Both furosemide and low-[Cl⁻]ₒ treatments have been shown to block activity-driven glial cell swelling (Kimelberg & Frangakis, 1985; Ransom et al., 1985; Walz & Hinks, 1985).

Furosemide has been shown to block epileptiform activity in many standard laboratory seizure models tested. In rat hippocampal slices, these include (1) afterdischarge activity in CA1 elicited by tetanic Schaffer collateral stimulation, high potassium (high-K⁺(10 mm), both acute and prolonged bathing of slices in zero-magnesium medium, 4-aminopyridine (4-AP) (300 μm), bicuculline (100 μm), and zero-calcium (0-Ca+) (Hochman et al., 1995; Gutschmidt et al., 1999). Whole animal studies in rats showed that furosemide blocks kainic acid status in rats (Hochman et al., 1995; Schwartzkroin et al., 1998) and prevented sound-triggered seizures in audiogenic seizure-prone animals (Reid et al., 2000). Furosemide has also been shown to have antiepileptic effects in several studies on human subjects. Intravenously administered furosemide blocked spontaneously occurring interictal spiking and stimulation-evoked afterdischarges of the neocortex during intraoperative studies in patients with medically intractable seizures (Haglund & Hochman, 2005). In those studies, furosemide elicited profound antiepileptic effects on each subject regardless of their specific seizure type. A small clinical trial showed that furosemide significantly reduced seizure frequency in adults with refractory epilepsy (Ahmad et al., 1976).

Bumetanide, a more potent and specific antagonist of NKCC1 than furosemide, has also been studied in models of animal seizures. Bumetanide was found to be more potent than furosemide in blocking kainic acid-induced status in rats (Schwartzkroin et al., 1998), and in preventing sound-triggered seizures in audiogenic seizure-prone rats (Reid et al., 2000). Bumetanide was also found to be more potent than furosemide in blocking epileptiform activity generated by focal application of bicuculline or 4-AP to the primate cortex, as well as in blocking stimulation-evoked afterdischarges in primate cortex (Haglund & Hochman, 2009).

### SUMMARY

The treatment compositions and methods of the present invention are useful for treating psychiatric and neurological disorders, including the anxiety disorders (posttraumatic stress disorder, generalized anxiety disorder, panic disorder, obsessive compulsive disorder, specific phobia), epilepsy, and seizure disorders (American Psychiatric Association, Diagnostic and Statistical Manual of Mental Disorders, 4^{th} edtion—Text Revision, 2000), as well as migraine, sleep disorders, obesity, eating disorders, autism, depression, edema, glaucoma, stroke, ischemia, neuropathic pain, tinnitus, addictive disorders, schizophrenia, psychosis, and tinnitus. The inventive compositions and methods may be employed to treat these, as well as other neurological and psychiatric disorders, while avoiding the unwanted cognitive and neurological side effects often associated with agents currently employed for the treatment of these disorders. The methods and compositions disclosed herein generally involve the cation-chloride cotransporter families NKCC and/or KCC.

Analogs and prodrugs of CNS-targeted NKCC co-transporter antagonists bumetanide, furosemide and piretanide include those described below. The inventors believe that such analogs have increased lipophilicity and reduced diuretic effects compared to the loop diuretics from which they are derived, and thus result in fewer undesirable side effects when employed in the inventive treatment methods.

In one embodiment, the level of diuresis that occurs following administration of an effective amount of one of the analogs or prodrugs described below is less than 90%, 80%, 70%, 60%, 50%, 40%, 30%, 20%, or 10% of that which occurs following administration of an effective amount of the loop diuretic from which the analog or prodrug is derived. For example, the analog or prodrug may be less diuretic than the standard loop diuretic molecule (i.e. bumetanide, furosemide or piretanide), when administered at the same mg/kg dose. Alternatively or additionally, the analog or prodrug may be more potent than the standard loop diuretic molecule from which it is derived, so that a smaller dose of the analog or prodrug is required for effective relief of symptoms, thereby eliciting less of a diuretic effect. For some treatments and for some molecules, the analog or prodrug may have a longer duration of action of its therapeutic effects for treating disorders than the standard loop diuretic molecule from which it is derived, so that the analog or prodrug may be administered less frequently than the standard loop diuretic molecule, thus leading to a lower total diuretic effect within any given period of time.

The inventive treatment agents may be administered in combination with other known treatment agents, such as those presently used in the treatment of psychiatric disorders and/or epilepsy. One with skill in the art will appreciate that the combination of a treatment agent of the present invention with other known treatment agent(s) will positively affect a wider spectrum of therapeutic targets, thus providing a more efficacious therapeutic effect than would otherwise be possible.

In general, the treatment compositions and methods of the present invention may be used therapeutically and episodically following the onset of symptoms, or prophylactically prior to the onset of symptoms. For example, treatment agents of the present invention can be used to treat existing anxiety disorders, or to prevent the development of specific anxiety disorders, such as Post Traumatic Stress Disorder, in individuals entering or undergoing stressful situations that are known to trigger the development of such disorders (such as a soldier entering the battle field). The above-mentioned and additional features of the present invention, together with the manner of obtaining them, will be best understood by reference to the following more detailed description. All references disclosed herein are hereby incorporated by reference in their entirety as if each was incorporated individually.

### BRIEF DESCRIPTIONS OF DRAWINGS

FIGS. 1A and 1B (Working example) show the effects of furosemide and bumetanide on suppressing anxiety in two different rat models of anxiety. FIG. 1A shows experimental results using the fear potentiated startle anxiety model, and FIG. 1B shows experimental results using the contextual fear conditioning anxiety model.

### DETAILED DESCRIPTION

Several classes of compounds that are analogs and prodrugs of loop diuretics bumetanide, furosemide and piretanide and that are believed to be novel are disclosed below. A first class of compounds, identified by Formulas I, II and III below, includes 5-ester derivatives of loop diuretics, which are anticipated to act as prodrugs of bumetanide, furosemide and piretanide. The synthetic methods for the preparation of these compounds would be considered standard to those skilled in the art.

Formula I, II and III compounds are as follows:

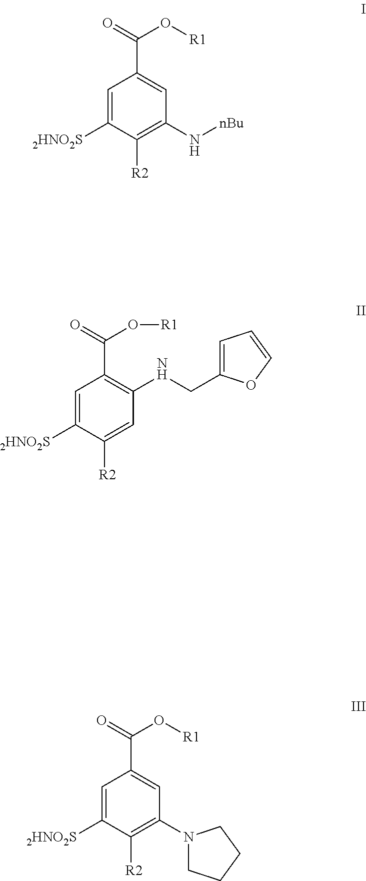

In various aspects, the present invention provides a compound having a structure according to formula I, II or III or a pharmaceutically acceptable salt, solvate or hydrate thereof, wherein

R1 is a member selected from substituted or unsubstituted cycloalkyl alkyl, substituted or unsubstituted alkylcarboxy alkyl, substituted or unsubstituted alkyldioxolone, substituted or unsubstituted alkylcarbonate alkyl, substituted or unsubstituted arylcarbonate alkyl, substituted or unsubstituted alkyloxycarbonyl alkyl, substituted or unsubstituted aryloxycarbonyl alkyl, alkyl acyl, aryl acyl, cycloalkyl acyl, heterocycloalkyl acyl, substituted or unsubstituted alkylphosphate alkyl, substituted or unsubstituted arylphosphate alkyl, substituted or unsubstituted aminoacid alkyl, substituted or unsubstituted cyclicaminoacid alkyl, substituted or unsubstituted bumetanide alkyl, substituted or unsubsittuted furosemide alkyl, and substituted or unsubsittuted piretanide alkyl;

R2 is member selected from halogen, trifluoromethyl, and XR3;

X is member selected from oxygen, sulfur, and nitrogen; and

R3 is member selected from hydrogen, alkyl, heteroalkyl, alkyltrifluoromethyl, aryl, heteroaryl, biphenyl and naphthalene.

Analogs of CNS-targeted NKCC co-transporter antagonists that may be usefully employed in the methods of the present invention further include 5-amido and 5-keto derivatives of bumetanide, furosemide and piretanide in which the 5-ester has been replaced by either an amide according to formulas IV, V and VI or a ketone according to formulas VII, VIII and IX.

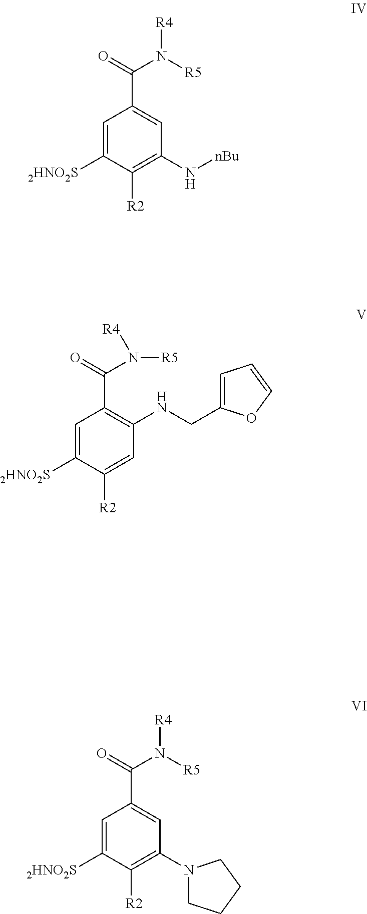

or a pharmaceutically acceptable salt, solvate or hydrate thereof, wherein:
R4 and R5 are independently:

R4 is a member selected from hydrogen, OR6, substituted or unsubstituted alkyl trifluoromethyl, substituted or unsubstituted alkynyl, substituted or unsubstituted alkynyl alkyl, substituted or unsubstituted amine dialkyl cycloalkyl alkyl, acyl, substituted or unsubstituted alkyl acyl, substituted or unsubstituted cycloalkyl acyl, substituted or unsubstituted amine dialkyl cycloalkyl acyl, substituted or unsubstituted heterocycloalkyl acyl, substituted or unsubstituted aryl acyl, substituted or unsubstituted heteroaryl acyl, substituted or unsubstituted cycloalkyl, substituted or unsubstituted cycloalkyl alkyl, substituted or unsubstituted heterocycloalkyl alkyl, substituted or unsubstituted alkyloxy alkyl, substituted or unsubstituted aryloxy alkyl, substituted or unsubstituted heteroaryloxy alkyl, substituted or unsubstituted cyclolalkyloxy alkyl, substituted or unsubstituted heterocycloalkyloxy alkyl, substituted or unsubstituted alkylthio alkyl, substituted or unsubstituted arylthio alkyl, substituted or unsubstituted heteroarylthio alkyl, substituted or unsubstituted cyclolalkylthio alkyl, or substituted or unsubstituted heterocycloalkylthio alkyl;

R5 is a member selected from hydrogen, OR6, substituted or unsubstituted alkyl trifluoromethyl, substituted or unsubstituted alkynyl, substituted or unsubstituted alkynyl alkyl, substituted or unsubstituted amine dialkyl cycloalkyl alkyl, acyl, substituted or unsubstituted alkyl acyl, substituted or unsubstituted cycloalkyl acyl, substituted or unsubstituted amine dialkyl cycloalkyl acyl, substituted or unsubstituted heterocycloalkyl acyl, substituted or unsubstituted aryl acyl, substituted or unsubstituted heteroaryl acyl, substituted or unsubstituted cycloalkyl, substituted or unsubstituted cycloalkyl alkyl, substituted or unsubstituted heterocycloalkyl alkyl, substituted or unsubstituted alkyloxy alkyl, substituted or unsubstituted aryloxy alkyl, substituted or unsubstituted heteroaryloxy alkyl, substituted or unsubstituted cyclolalkyloxy alkyl, substituted or unsubstituted heterocycloalkyloxy alkyl, substituted or unsubstituted alkylthio alkyl, substituted or unsubstituted arylthio alkyl, substituted or unsubstituted heteroarylthio alkyl, substituted or unsubstituted cyclolalkylthio alkyl, or substituted or unsubstituted heterocycloalkylthio alkyl;

R4 and R5, together with the nitrogen to which they are attached, form a saturated or unsaturated optionally substituted or unsubstituted bicyclic heterocyclic ring which may contain further heteroatoms, selected from oxygen, nitrogen or sulfur atoms; and

R6 is a member selected from hydrogen, substituted or unsubstituted alkyl, substituted or unsubstituted cycloalkyl, substituted or unsubstituted heterocycloalkyl, substituted or unsubstituted aryl, substituted or unsubstituted arylalkyl, and substituted or unsubstituted heteroarylalkyl;

R2 is member selected from halogen, trifluoromethyl, and XR3;

X is member selected from oxygen, sulfur, and nitrogen; and

R3 is member selected from hydrogen, alkyl, alkyltrifluoromethyl, aryl, and heteroaryl.

The present disclosure provides compounds having structures according to the formula VII, VIII, and IX:

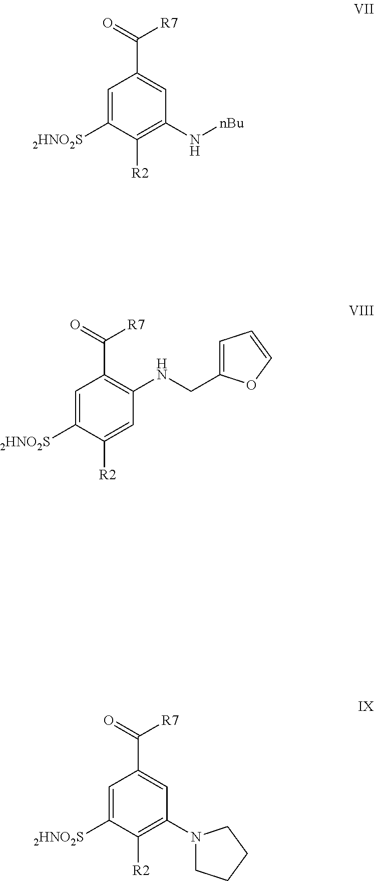

or a pharmaceutically acceptable salt, solvate or hydrate thereof, wherein:

R7 is a member selected from substituted or unsubstituted alkyl, substituted or unsubstituted cycloalkyl, substituted or unsubstituted heterocycloalkyl, substituted or unsubstituted arylalkyl, substituted or unsubstituted aryl, substituted or unsubstituted alkyloxyalkyl, substituted or unsubstituted alkyloxyaryl, substituted or unsubstituted alkyloxycycloalkyl, substituted or unsubstituted alkyloxyheteroaryl, substituted or unsubstituted alkylthioalkyl, substituted or unsubstituted alkylthioaryl, substituted or unsubstituted alkylthiocycloalkyl, substituted or unsubstituted alkylthioheteroaryl, substituted or unsubstituted alkylaminoalkyl, substituted or unsubstituted alkylaminoaryl, substituted or unsubstituted alkylaminocycloalkyl, substituted or unsubstituted alkylaminoheteroaryl, substituted or unsubstituted alkylcarboxyalkyl, substituted or unsubstituted alkylcarboxyaryl, substituted or unsubstituted alkylcarboxycycloalkyl, substituted or unsubstituted alkylcarboxyheteroaryl, substituted or unsubstituted alkyloxycarbonylalkyl, substituted or unsubstituted alkoxycarbonylaryl, substituted or unsubstituted alkoxycarbonylcycloalkyl, substituted or unsubstituted alkoxycarbonylheteroaryl, substituted or unsubstituted alkyltrifluoromethyl, and substituted or unsubstituted heteroarylalkyl;

R2 is member selected from halogen, trifluoromethyl, and XR3;

X is member selected from oxygen, sulfur, and nitrogen; and

R3 is member selected from hydrogen, alkyl, alkyltrifluoromethyl, aryl, and heteroaryl.

In yet additional aspects, the present invention provides compounds having the structures according to formulas X, XI and XII, shown below:

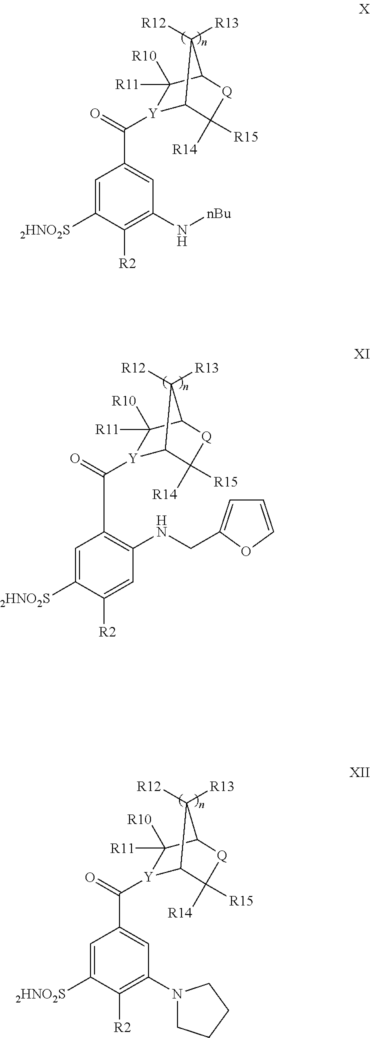

or a pharmaceutically acceptable salt, solvate or hydrate thereof, wherein:

n=1, 2;

Y is a member selected from nitrogen and CR8; and Q is a member selected from oxygen, sulfur, nitrogen and CR9;

R8 is hydrogen or alkyl; and

R9, R10, R11, R12, R13, R14, and R15, are each independently selected from the group consisting of: hydrogen, halogen, cyano, trifluoromethyl, alkyl, substituted or unsubstituted alkyl, substituted or unsubstituted cycloalkyl, substituted or unsubstituted heterocyclicalkyl, aryl, heteroaryl, substituted or unsubstituted arylalkyl, and substituted or unsubstituted heteroarylalkyl;

R2 is member selected from halogen, trifluoromethyl, and XR3;

X is member selected from oxygen, sulfur, and nitrogen; and

R3 is member selected from hydrogen, alkyl, alkyltrifluoromethyl, aryl, and heteroaryl.

According to still additional aspects, the present inventions provide compounds having structures according to the formula XIII, XIV, and XV:

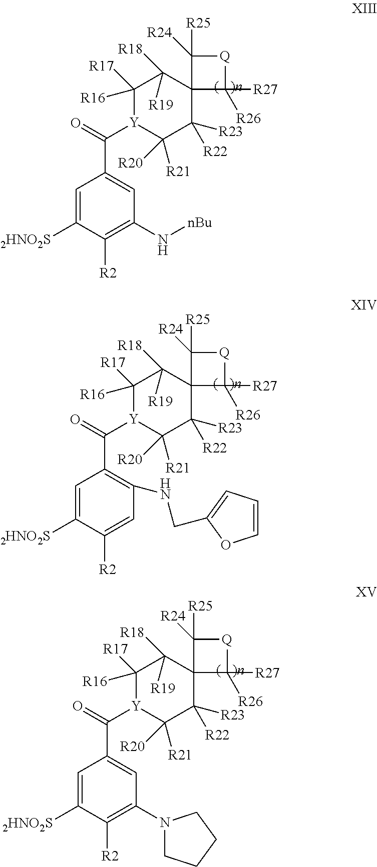

or a pharmaceutically acceptable salt, solvate or hydrate thereof, wherein:

n=1, 2, 3, 4

Y is a member selected from nitrogen and CR8; and Q is a member selected from oxygen, sulfur, nitrogen and CR9;

R8 is hydrogen or alkyl; and

R9, R16, R17, R18, R19, R20, R21, R22, R23, R24, R25, R26, and R27 are each independently selected from the group consisting of: hydrogen, halogen, cyano, trifluoromethyl, alkyl, substituted or unsubstituted alkyl, substituted or unsubstituted cycloalkyl, substituted or unsubstituted heterocyclicalkyl, aryl, heteroaryl, substituted or unsubstituted arylalkyl, and substituted or unsubstituted heteroarylalkyl.

R2 is member selected from halogen, trifluoromethyl, and XR3;

X is member selected from oxygen, sulfur, and nitrogen; and

R3 is member selected from hydrogen, alkyl, alkyltrifluoromethyl, aryl, and heteroaryl.

In still additional aspects, the present inventions provide compounds having structures according to the formula XVI, XVII, and XVIII:

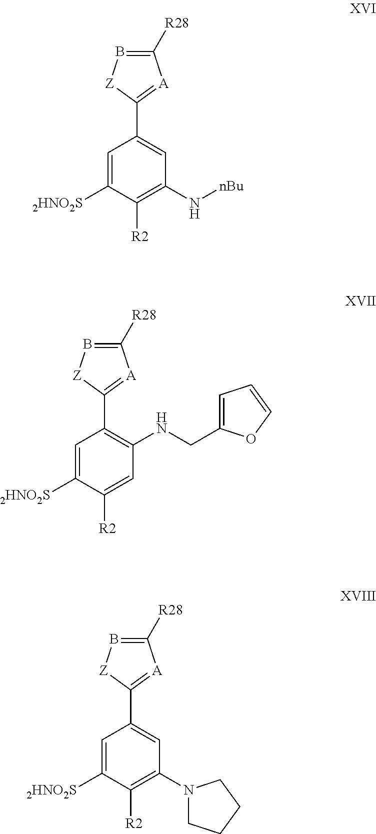

or a pharmaceutically acceptable salt, solvate or hydrate thereof, wherein:

Z is a member selected from oxygen, sulfur, nitrogen and CR29; A is a member selected from oxygen, sulfur, nitrogen and CR30, B is a member selected from oxygen, sulfur, nitrogen and CR31; and

R28, R29, R30, and R31 are each independently selected from the group consisting of: hydrogen, halogen, cyano, trifluoromethyl, substituted or unsubstituted alkyl, substituted or unsubstituted cycloalkyl, substituted or unsubstituted heterocyclicalkyl, substituted or unsubstituted aryl, substituted or unsubstituted heteroaryl, substituted or unsubstituted arylalkyl, and substituted or unsubstituted heteroarylalkyl.

R2 is member selected from halogen, trifluoromethyl, and XR3;

X is member selected from oxygen, sulfur, and nitrogen; and

R3 is member selected from hydrogen, alkyl, alkyltrifluoromethyl, aryl, and heteroaryl.

The compounds described in this invention can be synthesized using traditional synthesis techniques well known to those skilled in the art. More specific synthesis routes are outlined below.

Various ester-containing prodrugs such as compounds according to formula I, II, and III can be synthesized according to the schemes below:

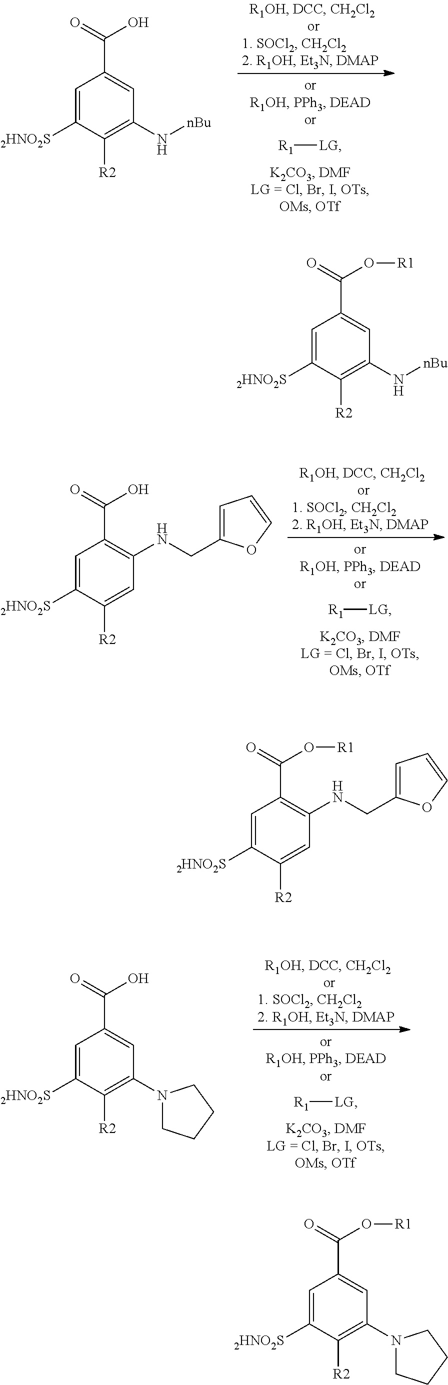

The Amide analogs can be synthesized according to the schemes below:

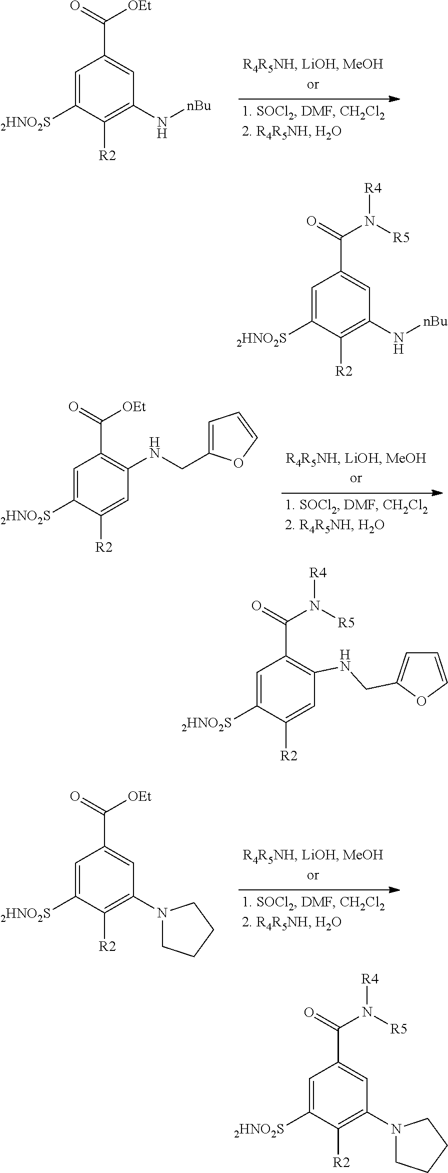

The synthesis for the Ketone versions of these derivatives would be as outlined in the following schemes:

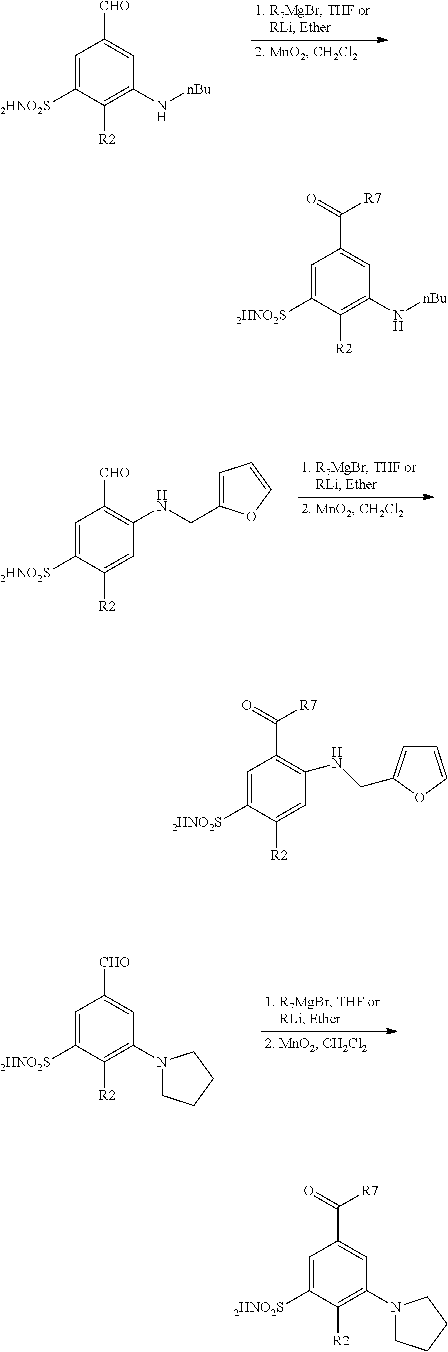

For the corresponding ketone analogs with the formulas X, XI, XII, XIII, XIV, and XV; they can be prepared according to the following schemes:

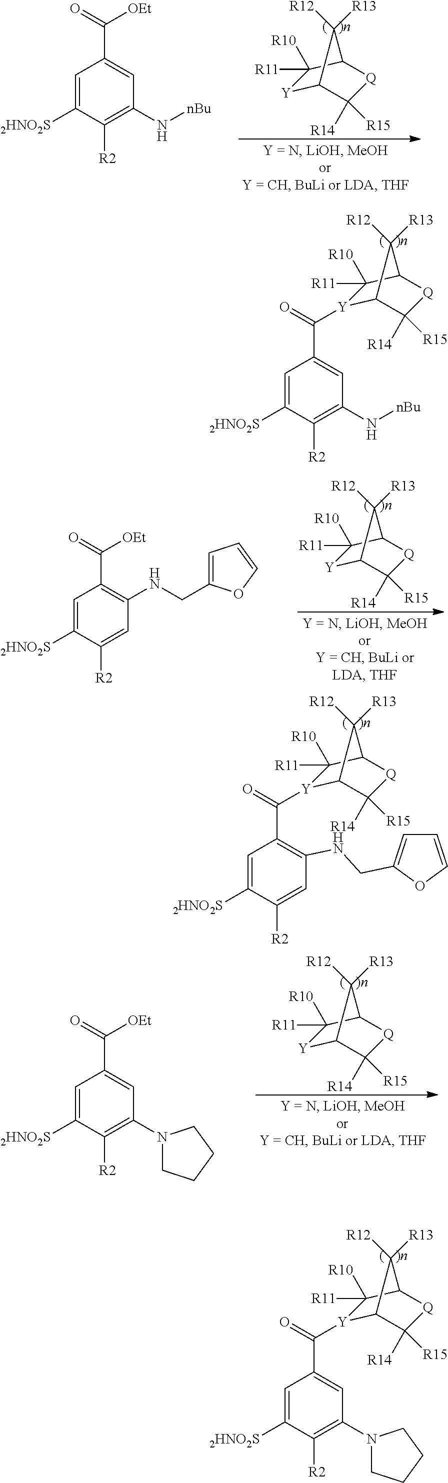

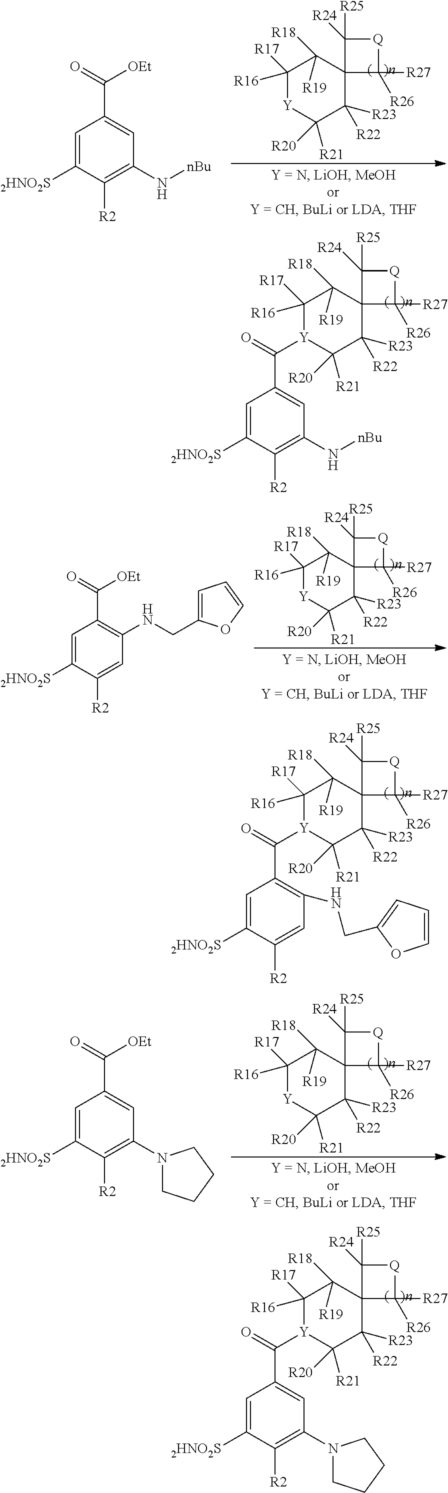

Synthetic preparation of the heterocyclic target compounds with the Formulas XVI, XVII, and XVIII can be achieved in three steps from the corresponding benzaldehydes as follows:

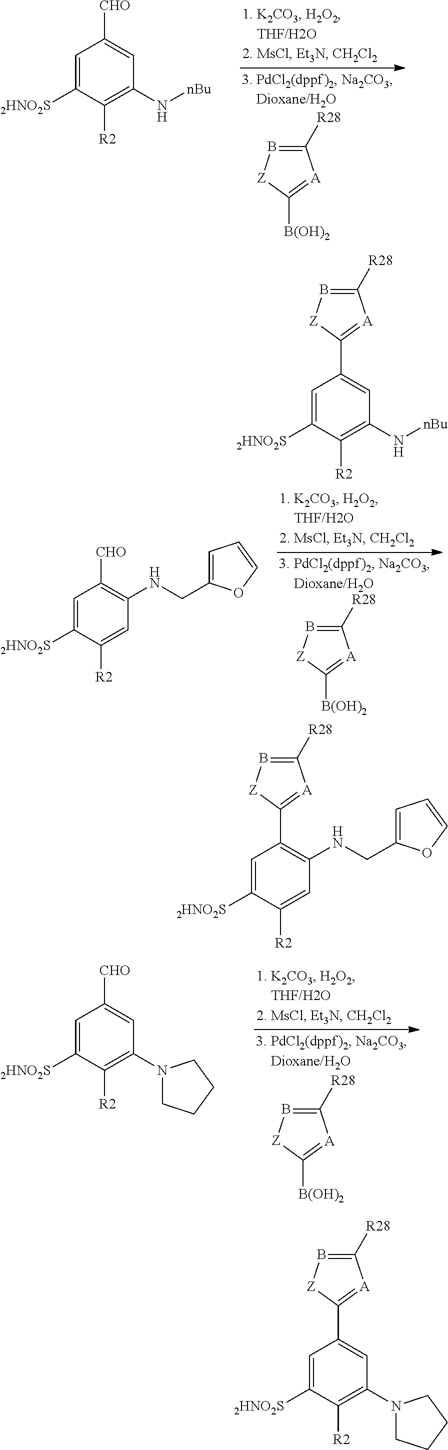

For many uses to treat diseases and conditions in humans, the above inventive analogs and prodrugs may be formulated in a capsule or gel-tabulate for oral delivery. The dose for inventive analogs and prodrugs would begin at ½ the dose of the common loop diuretic from which it is derived, and the dose could be increased to 10× beyond the standard dose, if necessary, since the inventive molecules would be substantially free from undesired side effects. For example, the inventive prodrugs and analogs of loop diuretics could be administered to adults in 0.25 mg doses, 2× per day, and increased up to 10 mg doses delivered 2× per day.

Pharmaceutical compositions of the present invention may be formulated, as is well known in the art, for oral, rectal, topical, nasal, inhalation (e.g, via an aerosol), vaginal, topical, transdermal and parenteral administration. Formulation of combinations of one or more active compounds with suitable carriers, stabilizers, and the like, to provide pharmaceutical compositions is within the skill in the art. In some applications, treatment compositions may be delivered in liposome formulations, for example, that cross the blood brain barrier, or may be co-administered with other agents that cross the blood brain barrier.

### EXAMPLE 1

### The Effects of Standard Loop Diuretics (Furosemide and Bumetanide) on Rat Models of Anxiety:

Methods:

Animal Handling and Drug Delivery

Ninety-six male, adult (3-4 months old) Long-Evans rats, housed in the University of Lethbridge vivarium, were used for these studies. Rat housing consisted of Plexiglas cages with sawdust bedding shared with two or three individuals. The colony room was temperature-controlled (20-21° C.) with a 12 h light/12 h dark cycle, beginning each day at 07:00. Food and water were provided ad libitum. Seventy-two hours prior to the experiment, rats were anaesthetized with isoflurane, and a cannula was implanted into the right external jugular vein of each rat for the purpose of administration of drugs [41]. Rats were thereafter kept in independent cages, and the cannulas were flushed daily to ensure patency. Bumetanide and furosemide were dissolved in DMSO (vehicle), and all drugs were administered I.V. via a cannulated jugular vein. Test drugs were administered 30 min prior to testing. All behavioural testing was conducted during the light cycle (7:00 am-7:00 pm). Testing occurred between the hours of 9:00 am and 3:00 pm. Different, randomly selected rats were used for each group (i.e. no rat was retested in more than one group). All testing was done under ambient room light.

Contextual Fear-Conditioning

Contextual Fear-Conditioning, following a previously described standard protocol, was performed on 24 rats [42]. The testing chamber consisted of a rectangular box (40 cm×56 cm×28 cm) with a stainless steel rod floor. All aspects of the timing of events were under microcomputer control (MedPC, MedAssociates Inc, Vermont, USA). Measurement of freezing was accomplished through an overhead video camera connected to a microcomputer and was automatically scored using a specialty piece of software, FreezeFrame. In Phase 1, rats were placed individually into the chambers for 5 minutes. Phase 2 occurred 24 hr later, when again rats were placed individually into the same chambers, they received an immediate (within 3 s of being placed into the chamber) foot shock (1 mA for 2 s). Thirty seconds later they were removed from the chambers. During phase 3, 24 hr later, the rats were returned to the chambers for 5 min. This session was video recorded and the amount of time spent freezing was assessed using FreezeFrame software. Freezing was defined as the total lack of body movement except for movement related to respiration. The percentage time spent freezing during each minute was entered into Excel spreadsheets and was analyzed using SPSS statistical software. One-way analysis of variance (ANOVA) was used to evaluate treatment effects.

Fear-Potentiated Startle

A Fear-Potentiated Startle protocol, following a previously described protocol, was used to test 23 rats [43]. Animals were trained and tested in four identical stabilimeter devices (Med-Associates). Each rat was placed in a small Plexiglas cylinder. The floor of each stabilimeter consisted of four 6 -mm-diameter stainless steel bars spaced 18 mm apart through which shock can be delivered. Cylinder movements result in displacement of an accelerometer where the resultant voltage is proportional to the velocity of the cage displacement. Startle amplitude was defined as the maximum accelerometer voltage that occurs during the first 0.25 sec after the startle stimulus was delivered. The analog output of the accelerometer was amplified, digitized on a scale of 0-4096 units and stored on a microcomputer. Each stabilimeter was enclosed in a ventilated, light-, and sound-attenuating box. All sound level measurements were made with a Precision Sound Level Meter. The noise of a ventilating fan attached to a sidewall of each wooden box produces an overall background noise level of 64 dB. The startle stimulus was a 50 ms burst of white noise (5 ms rise−decay time) generated by a white noise generator. The visual conditioned stimulus was the illumination of a light bulb adjacent to the white noise source. The unconditioned stimulus was a 0.6 mA foot shock with duration of 0.5 s, generated by four constant-current shockers located outside the chamber. The presentation and sequencing of all stimuli were controlled by computer. FPS procedures consist of 5 days of testing; during days 1 and 2 baseline startle responses were collected, days 3 and 4 light/shock pairings were delivered, day 5 testing for fear potentiated startle was conducted. Animals received treatment with compound or vehicle on days 3, 4, and 5.

Matching.

On days 1 and 2 rats were placed individually into the Plexiglas cylinders and 3 min later presented with 30 startle stimuli at a 30 sec interstimulus interval. An intensity of 105 dB was used. The mean startle amplitude across the 30 startle stimuli on the second day was used to assign rats into treatment groups with similar means.

Training.

On days 3 and 4, rats were placed individually into the Plexiglas cylinders. During the first 3 min in the chamber the rats were allowed to acclimate then 10 CS-shock pairings were delivered. The shock was delivered during the last 0.5 sec of the 3.7 sec CSs at an average intertrial interval of 4 min (range, 3-5 min)

Testing.

On the 5th day, rats were placed in the same startle boxes where they were trained and after 3 min acclimation were presented with 18 startle-eliciting stimuli (all at 105 dB). These initial startle stimuli were used to again habituate the rats to the acoustic startle stimuli. Thirty seconds after the last of these stimuli, each animal receives 60 startle stimuli with half of the stimuli presented alone (startle alone trials) and the other half presented 3.2 sec after the onset of the 3.7 sec CS (CS-startle trials). All startle stimuli were presented at a mean 30 sec interstimulus interval, randomly varying between 20 and 40 sec. Data were entered into Excel spreadsheets and SPSS for data analysis. Independent sample t-tests are used to compare each treatment groups.

Contextual Fear-Conditioning

The rats treated with bumetanide (N=8) and furosemide (N=8) spent a significantly smaller percentage of the test period freezing compared to the rats treated with vehicle alone (N=8) (vehicle mean=66.914 [SE=7.04]; bumetanide mean=24.3 [SE=6.80]; furosemide mean=30.12 [SE=4.91]) (df=2; F=13.382; p<0.0001).

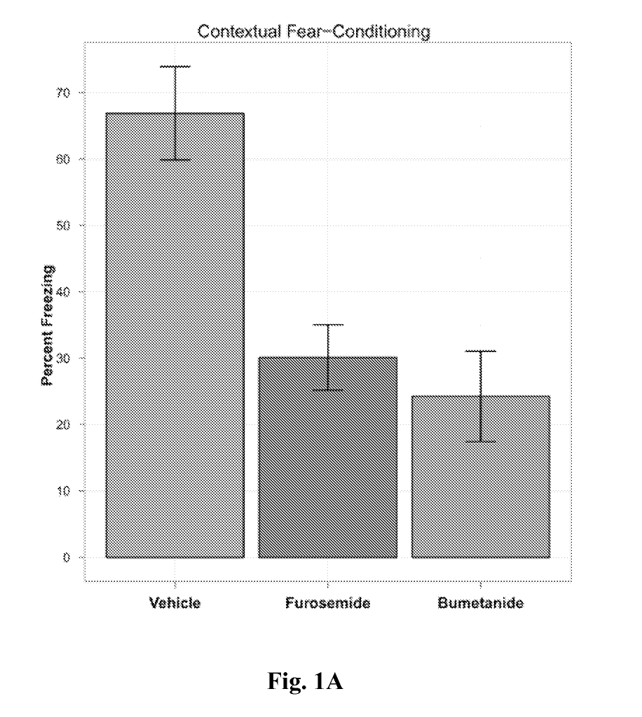

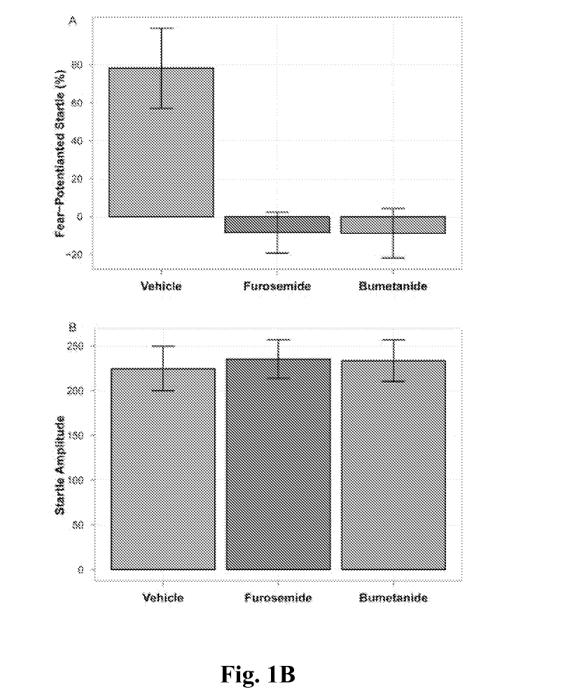

### FIG. 1A. Contextual Fear-Conditioning Results.

FIG. 1A shows the percentage of time during the contextual fear-conditioning test period during which rats were freezing, following intravenous injections of vehicle (N=8), bumetanide (N=8), and furosemide (N=8). Note: Error bars indicate standard errors.

Fear-Potentiated Startle

The rats treated with bumetanide (N=8) and furosemide (N=7) had significantly less increase in startle amplitude with the shock-conditioned stimulus than rats treated with vehicle alone (N=8) (vehicle mean=78.22 [SE=21.10]; bumetanide mean=−8.75 [SE=13.03]; furosemide mean=−8.42 [SE=10.82]) (df=2; F=9.99; p<0.001).

### FIG. 1B. Fear-Potentiated Startle Test Results.

FIG. 1B shows the startle amplitudes for rats receiving intravenous injections of vehicle (N=7), rats receiving furosemide (N=8), and rats receiving bumetanide (N=8). (A) Percent amount of fear-potentiated startle, and (B) amplitude of startle to the noise alone. Note: Error bars indicate standard errors.

We claim:

1. A compound comprising a 5-ester derivative of a loop diuretic.

2. A compound of claim 1 having a structure according to one of formulas I, II or III below: 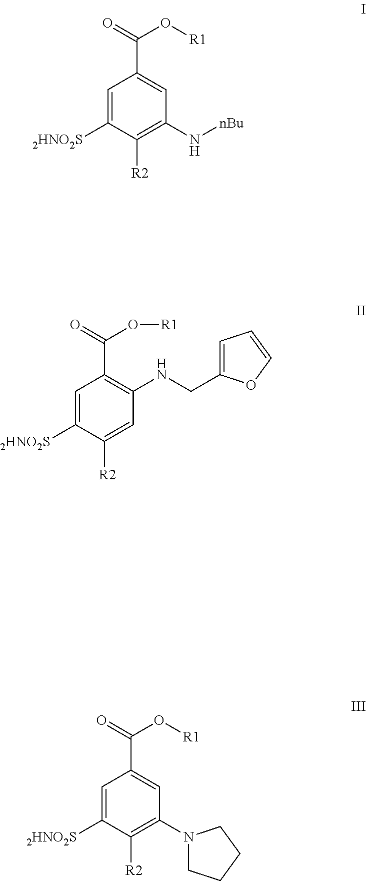
or a pharmaceutically acceptable salt, solvate or hydrate thereof, wherein
R1 is a member selected from substituted or unsubstituted cycloalkyl alkyl, substituted or unsubstituted alkylcarboxy alkyl, substituted or unsubstituted alkyldioxolone, substituted or unsubstituted alkylcarbonate alkyl, substituted or unsubstituted arylcarbonate alkyl, substituted or unsubstituted alkyloxycarbonyl alkyl, substituted or unsubstituted aryloxycarbonyl alkyl, alkyl acyl, aryl acyl, cycloalkyl acyl, heterocycloalkyl acyl, substituted or unsubstituted alkylphosphate alkyl, substituted or unsubstituted arylphosphate alkyl, substituted or unsubstituted aminoacid alkyl, substituted or unsubstituted cyclicaminoacid alkyl, substituted or unsubstituted bumetanide alkyl, substituted or unsubsittuted furosemide alkyl, and substituted or unsubsittuted piretanide alkyl;
R2 is member selected from halogen, trifluoromethyl, and XR3;
X is member selected from oxygen, sulfur, and nitrogen; and
R3 is member selected from hydrogen, alkyl, heteroalkyl, alkyltrifluoromethyl, aryl, heteroaryl, biphenyl and naphthalene.

3. A compound comprising a 5-amido or 5-keto derivative of a loop diuretic in which the 5-ester has been replaced by either a ketone or an amide.

4. A compound of claim 3 having a structure according to one of Formulas IV, V or VI, below: 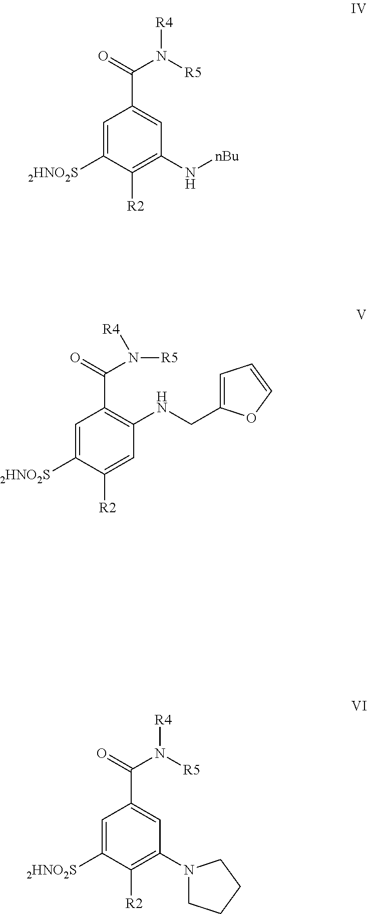
or a pharmaceutically acceptable salt, solvate or hydrate thereof, wherein:
R4 and R5 are independently:
R4 is a member selected from hydrogen, OR6, substituted or unsubstituted alkyl trifluoromethyl, substituted or unsubstituted alkynyl, substituted or unsubstituted alkynyl alkyl, substituted or unsubstituted amine dialkyl cycloalkyl alkyl, acyl, substituted or unsubstituted alkyl acyl, substituted or unsubstituted cycloalkyl acyl, substituted or unsubstituted amine dialkyl cycloalkyl acyl, substituted or unsubstituted heterocycloalkyl acyl, substituted or unsubstituted aryl acyl, substituted or unsubstituted heteroaryl acyl, substituted or unsubstituted cycloalkyl, substituted or unsubstituted cycloalkyl alkyl, substituted or unsubstituted heterocycloalkyl alkyl, substituted or unsubstituted alkyloxy alkyl, substituted or unsubstituted aryloxy alkyl, substituted or unsubstituted heteroaryloxy alkyl, substituted or unsubstituted cyclolalkyloxy alkyl, substituted or unsubstituted heterocycloalkyloxy alkyl, substituted or unsubstituted alkylthio alkyl, substituted or unsubstituted arylthio alkyl, substituted or unsubstituted heteroarylthio alkyl, substituted or unsubstituted cyclolalkylthio alkyl, or substituted or unsubstituted heterocycloalkylthio alkyl;
R5 is a member selected from hydrogen, OR6, substituted or unsubstituted alkyl trifluoromethyl, substituted or unsubstituted alkynyl, substituted or unsubstituted alkynyl alkyl, substituted or unsubstituted amine dialkyl cycloalkyl alkyl, acyl, substituted or unsubstituted alkyl acyl, substituted or unsubstituted cycloalkyl acyl, substituted or unsubstituted amine dialkyl cycloalkyl acyl, substituted or unsubstituted heterocycloalkyl acyl, substituted or unsubstituted aryl acyl, substituted or unsubstituted heteroaryl acyl, substituted or unsubstituted cycloalkyl, substituted or unsubstituted cycloalkyl alkyl, substituted or unsubstituted heterocycloalkyl alkyl, substituted or unsubstituted alkyloxy alkyl, substituted or unsubstituted aryloxy alkyl, substituted or unsubstituted heteroaryloxy alkyl, substituted or unsubstituted cyclolalkyloxy alkyl, substituted or unsubstituted heterocycloalkyloxy alkyl, substituted or unsubstituted alkylthio alkyl, substituted or unsubstituted arylthio alkyl, substituted or unsubstituted heteroarylthio alkyl, substituted or unsubstituted cyclolalkylthio alkyl, or substituted or unsubstituted heterocycloalkylthio alkyl;
R4 and R5, together with the nitrogen to which they are attached, form a saturated or unsaturated optionally substituted or unsubstituted bicyclic heterocyclic ring which may contain further heteroatoms, selected from oxygen, nitrogen or sulfur atoms, and
R6 is a member selected from hydrogen, substituted or unsubstituted alkyl, substituted or unsubstituted cycloalkyl, substituted or unsubstituted heterocycloalkyl, substituted or unsubstituted aryl, substituted or unsubstituted arylalkyl, and substituted or unsubstituted heteroarylalkyl.
R2 is member selected from halogen, trifluoromethyl, and XR3
X is member selected from oxygen, sulfur, and nitrogen
R3 is member selected from hydrogen, alkyl, alkyltrifluoromethyl, aryl, and heteroaryl.

5. A compound of claim 3 having a structure according to formula VII, VIII or IX, below: 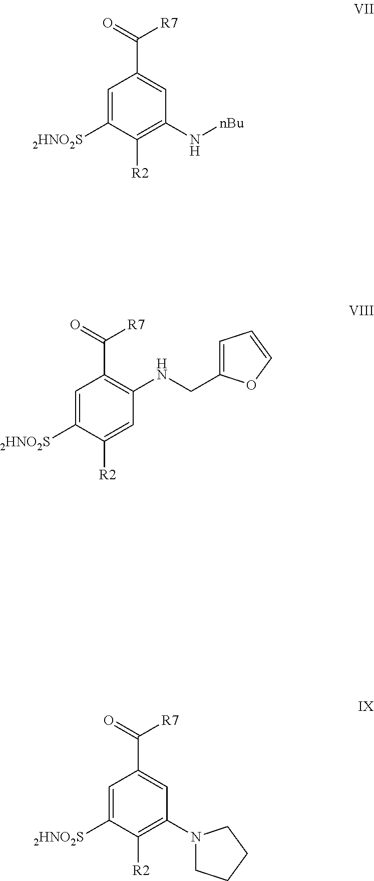
or a pharmaceutically acceptable salt, solvate or hydrate thereof, wherein:
R7 is a member selected from substituted or unsubstituted alkyl, substituted or unsubstituted cycloalkyl, substituted or unsubstituted heterocycloalkyl, substituted or unsubstituted arylalkyl, substituted or unsubstituted aryl, substituted or unsubstituted alkyloxyalkyl, substituted or unsubstituted alkyloxyaryl, substituted or unsubstituted alkyloxycycloalkyl, substituted or unsubstituted alkyloxyheteroaryl, substituted or unsubstituted alkylthioalkyl, substituted or unsubstituted alkylthioaryl, substituted or unsubstituted alkylthiocycloalkyl, substituted or unsubstituted alkylthioheteroaryl, substituted or unsubstituted alkylaminoalkyl, substituted or unsubstituted alkylaminoaryl, substituted or unsubstituted alkylaminocycloalkyl, substituted or unsubstituted alkylaminoheteroaryl, substituted or unsubstituted alkylcarboxyalkyl, substituted or unsubstituted alkylcarboxyaryl, substituted or unsubstituted alkylcarboxycycloalkyl, substituted or unsubstituted alkylcarboxyheteroaryl, substituted or unsubstituted alkyloxycarbonylalkyl, substituted or unsubstituted alkoxycarbonylaryl, substituted or unsubstituted alkoxycarbonylcycloalkyl, substituted or unsubstituted alkoxycarbonylheteroaryl, substituted or unsubstituted alkyltrifluoromethyl, and substituted or unsubstituted heteroarylalkyl;
R2 is member selected from halogen, trifluoromethyl, and XR3;
X is member selected from oxygen, sulfur, and nitrogen; and
R3 is member selected from hydrogen, alkyl, alkyltrifluoromethyl, aryl, and heteroaryl.

6. A compound having a structure according to formula X, XI or XII, below: 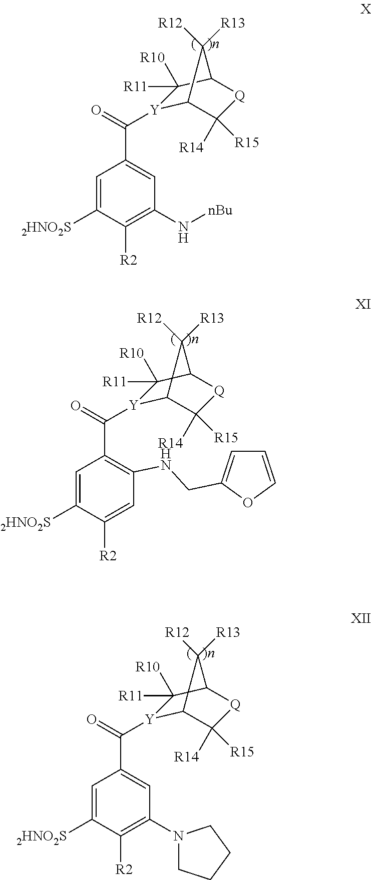
or a pharmaceutically acceptable salt, solvate or hydrate thereof, wherein:
n=1,2;
Y is a member selected from nitrogen and CR8; and Q is a member selected from oxygen, sulfur, nitrogen and CR9;
R8 is hydrogen or alkyl; and
R9, R10, R11, R12, R13, R14, and R15, are each independently selected from the group consisting of: hydrogen, halogen, cyano, trifluoromethyl, alkyl, substituted or unsubstituted alkyl, substituted or unsubstituted cycloalkyl, substituted or unsubstituted heterocyclicalkyl, aryl, heteroaryl, substituted or unsubstituted arylalkyl, and substituted or unsubstituted heteroarylalkyl.
R2 is member selected from halogen, trifluoromethyl, and XR3
X is member selected from oxygen, sulfur, and nitrogen
R3 is member selected from hydrogen, alkyl, alkyltrifluoromethyl, aryl, and heteroaryl.

7. A compound having a structure according to formula XIII, XIV or XV, below: 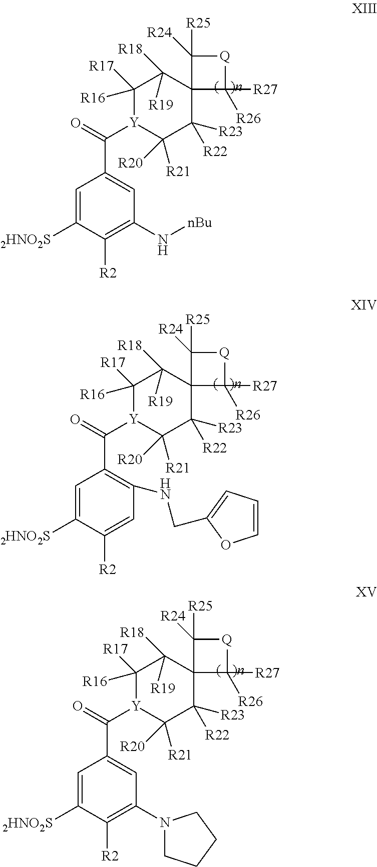
or a pharmaceutically acceptable salt, solvate or hydrate thereof, wherein:
n=1, 2, 3, 4
Y is a member selected from nitrogen and CR8; and Q is a member selected from oxygen, sulfur, nitrogen and CR9;
R8 is hydrogen or alkyl; and
R9, R16, R17, R18, R19, R20, R21, R22, R23, R24, R25, R26, and R27 are each independently selected from the group consisting of: hydrogen, halogen, cyano, trifluoromethyl, alkyl, substituted or unsubstituted alkyl, substituted or unsubstituted cycloalkyl, substituted or unsubstituted heterocyclicalkyl, aryl, heteroaryl, substituted or unsubstituted arylalkyl, and substituted or unsubstituted heteroarylalkyl.
R2 is member selected from halogen, trifluoromethyl, and XR3
X is member selected from oxygen, sulfur, and nitrogen
R3 is member selected from hydrogen, alkyl, alkyltrifluoromethyl, aryl, and heteroaryl.

8. A compound having a structure according to formula XVI, XVII or XVIII below: 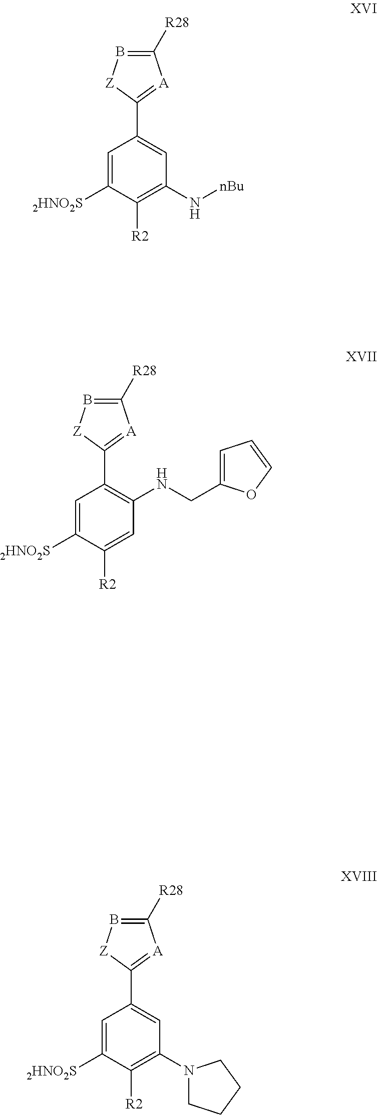
or a pharmaceutically acceptable salt, solvate or hydrate thereof, wherein:
Z is a member selected from oxygen, sulfur, nitrogen and CR29; A is a member selected from oxygen, sulfur, nitrogen and CR30, B is a member selected from oxygen, sulfur, nitrogen and CR31; and
R28, R29, R30, and R31 are each independently selected from the group consisting of: hydrogen, halogen, cyano, trifluoromethyl, substituted or unsubstituted alkyl, substituted or unsubstituted cycloalkyl, substituted or unsubstituted heterocyclicalkyl, substituted or unsubstituted aryl, substituted or unsubstituted heteroaryl, substituted or unsubstituted arylalkyl, and substituted or unsubstituted heteroarylalkyl.
R2 is member selected from halogen, trifluoromethyl, and XR3
X is member selected from oxygen, sulfur, and nitrogen
R3 is member selected from hydrogen, alkyl, alkyltrifluoromethyl, aryl, and heteroaryl.

9. A method for the treatment and/or prophylaxis of a neurological or psychiatric disorder comprising administering a composition having one of formulas I-XVIII disclosed herein.

10. The method of claim 9, wherein the neurological or psychiatric disorder is selected from the group consisting of: anxiety disorders (including posttraumatic stress disorder, generalized anxiety disorder, panic disorder, obsessive compulsive disorder, specific phobia), epilepsy, migraine, seizure disorders and non-epileptic seizures, sleep disorders, obesity, eating disorders, autism, depression, edema, glaucoma, stroke, ischemia, neuropathic pain, addictive disorders, schizophrenia, psychosis, and tinnitus.

11. The method of claim 9, comprising administering the composition following the onset of symptoms.

12. The method of claim 9, comprising administering the composition prophylactically prior to the onset of symptoms.

13. The method of claim 9, wherein the composition is formulated for oral delivery.

14. The method of claim 9, wherein the composition comprises a compound having a structure according to formula I, II or III.

15. The method of claim 9, wherein the composition comprises a 5-amido or 5-keto loop diuretic derivative in which the 5-ester has been replaced by either a ketone or an amide.

16. The method of claim 9, wherein the composition comprises a compound having a structure according to Formula IV, V or VI.

17. The method of claim 9, wherein the composition comprises a compound having a structure according to Formula VII, VIII or IX.

18. The method of claim 9, wherein the composition comprises a compound having a structure according to Formula X, XI or XII.

19. The method of claim 9, wherein the composition comprises a compound having a structure according to Formula XIII, XIV or XV.

20. The method of claim 9, wherein the composition comprises a compound having a structure according to Formula XVI, XVII or XVIII.
